(19) United States
(12) Patent Application Publication
(10) Pub. No.: US 2005/0079615 A1
(43) Pub. Date: Apr. 14, 2005

(54) Non-viral linear DNA vectors and methods for using the same

(76) Inventors: Mark A. Kay, Los Altos, CA (US); Zhi-Ying Chen, Mountain View, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/351,471

(22) Filed: Jan. 23, 2003

Related U.S. Application Data
(63) Continuation-in-part of application No. PCT/US01/23457, filed on Jul. 24, 2001.
(60) Provisional application No. 60/220,989, filed on Jul. 25, 2000.
(60) Provisional application No. 60/220,797, filed on Jul. 25, 2000.

(51) Int. Cl. A61K 48/00; C12N 15/867

(52) U.S. Cl. 435/456; 424/93.2

(57) ABSTRACT

Methods are provided for the in vivo introduction of a nucleic acid into the target cell of a vascularized organism, e.g., a mammal. In the subject methods, an aqueous formulation of a non-viral linear DNA vector, e.g., made up of a linear dsDNA molecule or non-annealed plus and minus linear ssDNA moelcules, that includes the nucleic acid is administered into the vascular system of the organism. Also provided are the vectors employed in the subject methods and kits for producing the same, as well as pharmaceutical preparations thereof. The subject methods and compositions find use in a variety of different applications, including both research and therapeutic applications, and are particularly suited for use in the in vivo delivery of nucleic acids encoding protein products, particularly where persistent protein expression is desired without integration of the vector into the host genome.

### CROSS REFERENCE TO RELATED APPLICATIONS

This application is a continuation-in-part application of application Ser. No. PCT/US01/23457 filed Jul. 24, 2001 and designating the United States; which application claims priority pursuant to 35 U.S.C. § 119 (e) to the filing date of the U.S. Provisional Patent Application Ser. No. 60/220,989 filed Jul. 25, 2000 and U.S. Provisional Patent Application Ser. No. 60/220,797; the disclosures of which applications are herein incorporated by reference.

### INTRODUCTION

1. Field of the Invention

The field of this invention is molecular biology, particularly transformation and specifically vectors employed in transformation.

2. Background of the Invention

The introduction of an exogenous nucleic acid sequence (e.g., DNA) into a cell, a process known as “transformation,” plays a major role in a variety of biotechnology and related applications, including research, synthetic and therapeutic applications. Research applications in which transformation plays a critical role include the production of transgenic cells and animals. Synthetic applications in which transformation plays a critical role include the production of peptides and proteins. Therapeutic applications in which transformation plays a key role include gene therapy applications. Because of the prevalent role transformation plays in the above and other applications, a variety of different transformation protocols have been developed.

In many transformation applications, it is desirable to introduce the exogenous DNA in a manner such that it provides for long term expression of the protein encoded by the exogenous DNA. Long term expression of exogenous DNA is primarily achieved through incorporation of the exogenous DNA into a target cell's genome. One means of providing for genome integration is to employ a vector that is capable of homologous recombination. Techniques that rely on homologous recombination can be disadvantageous in that the necessary homologies may not always exist; the recombination events may be slow; etc. As such, homologous recombination based protocols are not entirely satisfactory.

Accordingly, alternative viral based transformation protocols have been developed, in which a viral vector is employed to introduce exogenous DNA into a cell and then subsequently integrate the introduced DNA into the target cell's genome. Viral based vectors finding use include retroviral vectors, e.g., Moloney murine leukemia viral based vectors. Other viral based vectors that find use include adenovirus derived vectors, HSV derived vectors, sindbis derived vectors, etc. While viral vectors provide for a number of advantages, their use is not optimal in many situations. Disadvantages associated with viral based vectors include immunogenicity, viral based complications, and the like.

Accordingly, there is continued interest in the development of additional methods of transforming cells with exogenous nucleic acids to provide for persistent, long term expression of an encoded protein. Of particular interest is the development of a non-viral in vivo nucleic acid transfer protocol and vector that provides for persistent protein expression without concomitant genome integration.

Relevant Literature

U.S. patents of interest include 5,985,847 and 5,922,687. Also of interest is WO/11092. Additional references of interest include: Wolff et al., “Direct Gene Transfer Into Mouse Muscle In Vivo,” Science (March 1990) 247: 1465-1468; Hickman et al., “Gene Expression Following Direct Injection of DNA Into Liver,” Hum. Gen. Ther. (December 1994) 5:1477-1483; and Acsadi et al., “Direct Gene Transfer and Expression Into Rat Heart In Vivo,” New Biol. (January 1991) 3:71-81.

### SUMMARY OF THE INVENTION

Methods are provided for the in vivo introduction of a nucleic acid into the target cell of a vascularized organism, e.g., a mammal. In the subject methods, an aqueous formulation of a non-viral linear DNA vector that includes the nucleic acid is administered into the vascular system of the organism. The non-viral linear DNA vector may be either a linear dsDNA molecule or non-annealed plus and minus ssDNA molecules that are capable of hybridizing to produce the desired linear dsDNA molecule. Also provided are the vectors employed in the subject methods and kits for producing the same. The subject methods and compositions find use in a variety of different applications, including both research and therapeutic applications, and are particularly suited for use in the in vivo delivery of nucleic acids encoding protein products, particularly where persistent protein expression is desired without integration of the vector into the host genome.

### BRIEF DESCRIPTION OF THE DRAWINGS

FIG. 1 provides a schematic representation of a non-viral linear dsDNA vector according to the subject invention.

FIG. 2. Maps of hAAT constructs. Construct of pRSV.hAAT.bpA. Within the pBS.KS backbone, human alpha-1 antitrypsin (hAAT) cDNA with a poly A signal from bovine growth factor gene is placed under the control of Rous sarcoma virus long term repeat (RSV). Two linear DNA fragments, the RSV.hAAT.bpA expression cassette and the bacteria backbone, are produced by Xho I digestion and used as LDNA in this study. The uncut closed circular plasmid DNA (ccDNA) is used as control. pABsg.hAAT.bpA. This construct contains the same RSV.hAAT.bpA expression cassette as pRSV.hAAT.bpA. The only difference is that wild type adenovirus-associated virus (AAV) inverted terminal repeat (ITR), derived from the pALAPSN construct (Alexander IE et al., JV 68:8282, 1984), was built into the construct in a way that the hAAT expression cassette flanking with the wild type AAV ITR can be released by Bsg I digestion and, together with the bacteria backbone, used as AAV LDNA in this study. The undigested plasmid DNA (AAV ccDNA) was used as control DNA.

FIG. 3. HAAT expression in mice. (A), HAAT expression in mice receiving LDNA and ccDNA. Forty μg of LDNA and ccDNA in 2 ml of saline were given to each mouse via tail vein injection (Wolff J A et al., and Liu F et al.,). Mouse blood was collected periodly by retro-orbital bleeding and seral hAAT was determined by ELISA using a goat anti-hAAT antiby from DiaSorine (Stillwater, Minn.; Kay, M. et al., Hepatology, 815-819, 1995). To serve as a negative control, plasmid of pRSV.haat.bpA was digested with BsrD1 (Fragmented DNA), which destroyed the hAAT expression cassette by cutting it 3 times, and a same amount of the fragmented DNA was given to the mice. Seral hAAT was expressed as mean±S.E. ng/ml. (B), A repeat of the experiment desscribed in (A). This experiment was a repeat to confirm the observations in the first experiment. All experiment conditions were the same except that each group contained 6 mice until week 10 when 3 mice each were sacrificed for Southern blot analysis. A same amount of pBS.KS plasmid DNA was used in place of fragmented DNA as negative control. (C), HAAT expression in mice receiving AAV LDNA and AAV ccDNA. All experiment conditions were the same as the previous 2 experiments. The only difference was that wild type AAV ITR ws included in both AAV LDNA and AAV ccDNA (FIG. 2).

FIG. 4. Quantitation of vector DNA in mouse liver. (A), Southern blot demonstration of relative amount of vector DNA in mouse livers. Liver total DNA was prepared from livers of mice 1 day, 5 weeks, and 10 weeks after receiving 40 μg of vector DNA via tail vein injection. Twenty μg of liver DNA was digested with EcoR 1, which released the 1.35 kb hAAT cDNA fragment (FIG. 2), and separated in 0.8 percent of agarose gel. The band was visualized using the same Eco R1 released hAAT cDNA as probe. The upper panel was a series of standards composed of 400, 100, 25, 5 and 0 copies of the same 1.35 kb hAAT cDNA fragment spiked with 20 μg of liver genomic DNA from untreated mice, digested with Eco R1, and run in the same gel as the experimetnal DNA samples. The other panels are samples from mice injected with linear DNA (LDNA), linear DNA flanked with AAV ITR (AAV LDNA), closed circular plasmid (ccDNA), and ccDNA containing AAV ITR (AAV ccDNA), respectively. Three mice were used in all groups except day 1 and week 5 time points in AAV ccDNA group where only 1 animals were used. (B), Vector DNA copy number determined by phosphoimage protocol. The amount of vector DNA was expressed as mean±S.E. copies per mouse liver diploidy genome. Only the averages were shown for the day 1 and week 5 time points of AAV ccDNA group. * indicates statistically significant difference between groups at p<0.05.

FIG. 5 provides a schematic of formation of the large concatamers by 2 DNA fragments, produced by Xho I-digestion of pRSV.hAAT.bpA in mouse liver.

FIG. 6 provides a schematic representation of a vector according to the subject invention.

FIG. 7 provides a map of pA.sApoEHCR.phAAT.hFIX+intronA.bpA construct and schematic illustration of ssDNA preparation. In this figure, the box marked with EI stands for DNA sequence of exon 1 and a part of intron A of hFIX gene. Also shown in FIG. 7 is the production of ssDNA, where the phagemid ssDNA was heated to anneal and then digested with BsrD1 or NIavIV to produce ssDNA with or without AAV ITR.

FIGS. 8 and 9 show the serum hFIX concentrations in mice receiving AAV ssDNA. Forty μg of AAV ssDNA, either plus strand, minus strand alone or a mixture of both were dissolved in 2 ml of saline and delivered to 8 to 10 week old C57BL/6 mice via tail vein injection. Blood was periodically collected and the serum hFIX determined by ELISA.

FIG. 10 shows human FIX expression in mice receiving ssDNA without AAV ITR. The ssDNA used in this experiment was prepared by digestion of the phagemid ssDNA with NIaIV which produced the ssDNA of the hFIX expression cassette without AAV ITR.

FIG. 11 provides a schematic illustration showing the dynamic change of the 2 complementary strands of ssDNA in mouse liver and the results of Kpn I digestion of vector DNA in mouse liver. Kpn I cut once through the expression cassette.

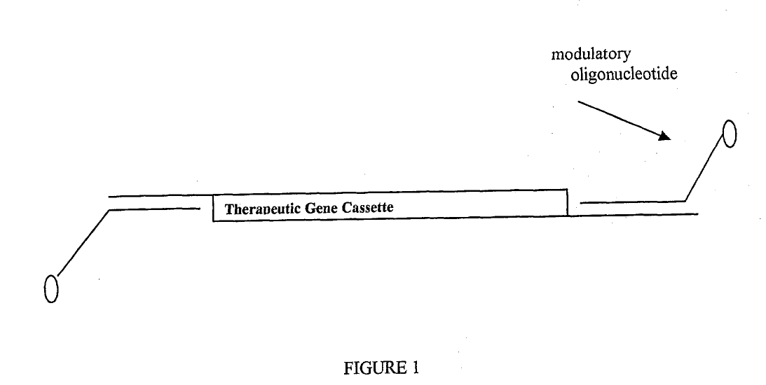

### DESCRIPTION OF THE SPECIFIC EMBODIMENTS

Methods are provided for the in vivo introduction of a nucleic acid into the target cell of a vascularized organism, e.g., a mammal. In the subject methods, an aqueous formulation of a non-viral linear DNA vector (either a single stranded or double stranded vector) that includes the nucleic acid is administered into the vascular system of the organism. Also provided are the vectors employed in the subject methods and kits for producing the same. The subject methods and compositions find use in a variety of different applications, including both research and therapeutic applications, and are particularly suited for use in the in vivo delivery of nucleic acids encoding protein products, particularly where persistent protein expression is desired without integration of the vector into the host genome. In further describing the invention, the subject methods will be described first followed by a description of representative applications in which the subject methods find use.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Methods

As summarized above, the present invention provides methods of introducing an exogenous nucleic acid into at least the nucleus of at least one cell, i.e., a target cell, of a multicellular organism. In many embodiments, the present invention provides methods of introducing an exogenous nucleic acid into the nucleus of a plurality of the cells of the host, whereby plurality is often meant at least about 0.1 number %, usually at least about 0.5 number % in certain embodiments. A feature of the subject invention is that the subject methods are in vivo methods, by which is meant that the exogenous nucleic acid is administered directly to the multicellular organism, in contrast to in vitro methods in which the target cell or cells are removed from the multicellular organism and then contacted with the exogenous nucleic acid. As specified below, the subject methods rely on systemic administration of the vector employed in the subject methods, where by systemic administration is meant that the vector is administered to the host in a manner such that it comes into contact with more than just a local area or region of the host, where by local area or region of the host is meant a region that is less than about 10%, usually less than about 5% of the total mass of the host.

A feature of the subject invention is that the methods employ a non-viral linear DNA vector. By “linear” is meant that the DNA vectors of the subject methods are not circular or closed vectors, but instead have a terminus at either end, where, depending on whether the vector is single or double stranded, the terminus may be blunt ended or staggered, i.e., sticky, have an overhang, etc. As such, the vectors are not plasmids. In many embodiments, the vectors are also characterized in that they are not complexed with a lipid component, e.g., a liposome, synthetic or naturally occurring lipid, etc. In certain embodiments, the vectors may be “naked” DNA in that they are not complexed with any other component, while in other embodiments they may be complexed with another component.

As indicated above, the vectors may be either double stranded or single stranded vectors. Each embodiment is now described separately in greater detail.

Double Stranded DNA (dsDNA) Vectors

The overall length of the linear dsDNA vector is sufficient to include the desired elements, as described below, but not so long as to prevent or substantially inhibit to an unacceptable level the ability of the vector to enter the target cell upon system administration to the host. As such, the linear dsDNA vector is generally at least about 0.5 kb long, usually at least about 4 kb long and more usually at least about 6 kb long, where the vector may be as long as 50 kb or longer, but in many embodiments does not exceed about 12 kb long and usually does not exceed about 20 kb long. In many embodiments, the length of the dsDNA vector ranges from about 0.5 to 20 kb, usually from about 4.0 to 12 kb.

As mentioned above, the dsDNA vector may be blunt ended or include a staggered end, in which one strand overhangs the other strand. The length of the overhang in these latter embodiments may vary, but in many embodiments ranges from about 1 to 11 bp, usually from about 2 to 6 bp.

Where the vector includes overhangs at either end, the opportunity arises to include a modulatory oligonucleotide. These modulatory oligonucucleotides are described in greater detail, infra.

Linear Single Stranded DNA (ssDNA) Vectors

The subject linear ssDNA vectors are characterized in that they are made up of non-annealed single stranded complementary plus (+) and (−) strands. As such, the subject vectors may also be viewed as vector systems made up of non-annealed but complementary nucleic acids, where each pair of complementary nucleic acids is made up of a plus and minus strand. As the two nucleic acids of each pair that make up a vector or vector system according to the subject invention are complementary, they hybridize to each other under stringent conditions, where representative stringent conditions are provided below in greater detail.

The overall length of the linear ssDNA molecules that make up the subject vectors is sufficient to include the desired elements as described above, but not so long as to prevent or substantially inhibit to an unacceptable level the ability of the vector to enter the target cell upon systemic administration to the host. As such, the linear ssDNA molecules of the subject vectors are generally at least about 100 bp long, usually at least about 500 bp long and more usually at least about 2000 bp long, where the vectors may be as long as 10,000 bp or longer, but in many embodiments do not exceed about 6 kb long. In many embodiments, the length of the plus and minus single strands of the subject vectors ranges from about 100 bases to about 50 kb, usually from about 1000 to 7000 bases.

In certain embodiments, the subject vectors include at least one modulatory oligonucleotide. Modulatory oligonucleotides are described in greater detail, infra.

Additional Vector Features

The subject linear DNA vectors may further include at least one restriction endonuclease recognized site, i.e., a restriction site. A variety of restriction sites are known in the art and may be included in the vector, where such sites include those recognized by the following restriction enzymes: HindIII, PstI, SalI, AccI, HincII, XbaI, BamHI, SmaI, XmaI, KpnI, SacI, EcoRI, and the like. In many embodiments, the vector includes a polylinker, i.e., a closely arranged series or array of sites recognized by a plurality of different restriction enzymes, such as those listed above. As such, in many embodiments, the vectors include a multiple cloning site made up of a plurality of restriction sites. The number of restriction sites in the multiple cloning site may vary, ranging anywhere from 2 to 15 or more, usually 2 to 10.

The DNA vectors include at least one nucleic acid of interest, i.e. a nucleic acid that is to be introduced into the target cell, e.g., to be expressed as protein in the target cell, etc., as described in greater detail below. The subject vectors may include a wide variety of nucleic acids, where the nucleic acids may include a sequence of bases that is endogenous and/or exogenous to the multicellular organism, where an exogenous sequence is one that is not present in the target cell while an endogenous sequence is one that pre-exists in the target cell prior to introduction. In any event, the nucleic acid of the vector is exogenous to the target cell, since it originates at a source other than the target cell and is introduce into the cell by the subject methods, as described infra. The nature of the nucleic acid will vary depending the particular protocol being performed. For example, in research applications the exogenous nucleic acid may be a novel gene whose protein product is not well characterized. In such applications, the vector is employed to stably introduce the gene into the target cell and observe changes in the cell phenotype in order to characterize the gene. Alternatively, in protein synthesis applications, the exogenous nucleic acid encodes a protein of interest which is to be produced by the cell. In yet other embodiments where the vector is employed, e.g., in gene therapy, the exogenous nucleic acid is a gene having therapeutic activity, i.e., a gene that encodes a product of therapeutic utility.

A variety of different features may be present in the vector. In many embodiments, the vector is characterized by the presence of at least one transcriptionally active gene. By transcriptionally active gene is meant a coding sequence that is capable of being expressed under intracellular conditions, e.g., a coding sequence in combination with any requisite expression regulatory elements that are required for expression in the intracellular environment of the target cell into which the vector is introduced by the subject methods. As such, the transcriptionally active genes of the subject vectors typically include a stretch of nucleotides or domain, i.e., expression module or expression cassette, that includes a coding sequence of nucleotides in operational combination, i.e. operably linked, with requisite trascriptional mediation or regulatory element(s). Requisite transcriptional mediation elements that may be present in the expression module include promoters, enhancers, termination and polyadenylation signal elements, splicing signal elements, and the like.

Preferably, the expression module or expression cassette includes transcription regulatory elements that provide for expression of the gene in a broad host range. A variety of such combinations are known, where specific transcription regulatory elements include: SV40 elements, as described in Dijkema et al., EMBO J. (1985) 4:761; transcription regulatory elements derived from the LTR of the Rous sarcoma virus, as described in Gorman et al., Proc. Nat'l Acad. Sci USA (1982) 79:6777; transcription regulatory elements derived from the LTR of human cytomegalovirus (CMV), as described in Boshart et al., Cell (1985) 41:521; hsp70 promoters, (Levy-Holtzman, R. and I. Schechter (Biochim. Biophys. Acta (1995) 1263: 96-98) Presnail, J. K. and M. A. Hoy, (Exp. Appl. Acarol. (1994) 18: 301-308)) and the like.

In certain embodiments, the at least one transcriptionally active gene or expression module present in the vector acts as a selectable marker. A variety of different genes have been employed as selectable markers, and the particular gene employed in the subject vectors as a selectable marker is chosen primarily as a matter of convenience. Known selectable marker genes include: the thimydine kinase gene, the dihydrofolate reductase gene, the xanthine-guanine phosporibosyl transferase gene, CAD, the adenosine deaminase gene, the asparagine synthetase gene, the antibiotic resistance genes, e.g. tet^{r}, amp^{r}, Cm^{r} or cat, kan^{r} or neo^{r} (aminoglycoside phosphotransferase genes), the hygromycin B phosphotransferase gene, genes whose expression provides for the presence of a detectable product, either directly or indirectly, e.g. β-galactosidase, GFP, and the like.

In many embodiments, the at least one transcriptionally active gene or module encodes a protein that has therapeutic activity for the multicellular organism, where such proteins include, but are not limited to: factor VIII, factor IX, β-globin, low-density protein receptor, adenosine deaminase, purine nucleoside phosphorylase, sphingomyelinase, glucocerebrosidase, cystic fibrosis transmembrane regulator, α-antitrypsin, CD-18, ornithine transcarbamylase, arginosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, interleukins, cytokines, small peptides etc, and the like. Of particular interest in many embodiment are coding sequences for the human versions of the above proteins.

Modulatory Oligonucleotides

As indicated above, the linear vector may further include one or more modulatory oligonucleotides, where the modulatory oligonucleotide modifies the properties of the vector in one or more desirable ways. For example, the modulatory oligonucleotide may target the vector to a particular cell or subcellular location, may modulate the half-life of the vector, may enhance its passage into a target cell, etc. Of particular interest are modulatory oligonucleotides that target the vector to a particular cellular or subcellular location upon administration to the host, i.e., targeting modulatory oligonucleotides.

In many embodiments, the modulatory oligonucleotides include an oligonucleotide domain stably attached to a ligand. By stably attached is meant that the oligonucleotide domain and ligand are associated with each other in a manner such that they do not readily dissociated from each other, even under washing conditions and/or physiologically conditions encountered inside the host. In many embodiments, the oligonucleotide domain and ligand are covalently attached, either directly or through a linking group.

The oligonucleotide domain is a domain having a sequence of nucleotides that is capable of hybridizing under stringent conditions, e.g., 50° C. or higher and 0.1×SSC (15 mM sodium chloride/1.5 mM sodium citrate)(or conditions analogous thereto), to a sequence of nucleotides found in the overhang of the dsDNA vector. This complementary sequence may vary in length so long as it is of sufficient length to provide for the desired hybridization under stringent conditions, where the length of this complementary sequence typically ranges from about 2 to 50 bp, usually from about 5 to 10 bp. The overall length of the oligonucleotide domain of the modulatory oligonucleotide may be the same or different, where when the oligonucleotide domain is longer than this complementary domain, the length of the oligonucleotide domain typically ranges from about 2 bp to 50 bp, usually from about 4 bp to 50 bp. The sequence of the oligonucleotide domain may be any convenient sequence, so long as it provides for the desired hybridization under stringent conditions to the overhang of the dsDNA vector.

The ligand component may be any convenient moiety that provides for the desired modulatory activity, e.g., targeting. The ligand may be a peptide, small organic molecule, or other convenient moiety that is capable of binding with sufficient affinity to a desired site inside the host, e.g., a receptor, target protein, etc. The size of the ligand may vary, where in many embodiments the ligand may range in size from about 100 daltons to 1000 kd, usually from about 5 kd to 50 kd. Of particular interest in certain embodiments are targeting peptides. Targeting peptides of interest for use as ligands in the subject modulatory oligonucleotides and dsDNA vectors including the same include, but are not limited to: asialoglycoproteins, tissue specific ligands, EGF receptors, nuclear location signals, endosomal lysis peptides; and the like.

Depending on whether the vector is a single stranded or double stranded molecule, one or more different or identical modulatory oligonucleotides may be employed. For example, where the subject dsDNA vectors include a hybridized modulatory oligonucleotide at either terminus, the two modulatory oligonucleotides may be the same or different with respect to the nature of the oligonucleotide domain and/or ligand, depending on the nature of the overhang, the desired modulatory properties, and the like. With respect to the ssDNA vectors, the subject vectors may include a single modulatory oligonucleotide, or a plurality of two or more different types of modulatory oligonucleotides, where there may or may not be correspondence or agreement between the modulatory sequence(s) hybridized to the plus strand and the minus strand that make up of the vector. In many embodiments, there is correspondence or agreement in the nature of the modulatory oligonucleotide(s) hybridized to each of the plus and minus strand, such that the same modulatory oligonucleotide(s) is hybridized to each of the plus and minus strands. In the above embodiments that include modulatory oligonucleotides, the plus and/or minus strands of the vector also include a sequence of nucleotides that is complementary to the complementary domain of the modulatory oligonucleotide, where this sequence is located 5′ and for 3′ of the expression cassette sequence and is of sufficient length to provide for hybridization to the desired number of modulatory oligonucleotides.

The above described dsDNA vectors may be produced using any convenient protocol. The procedures of cleavage, plasmid construction, cell transformation and plasmid production involved in many protocols employed to prepare the subject vectors are well known to one skilled in the art and the enzymes required for restriction and ligation are available commercially. (See, for example, R. Wu, Ed., Methods in Enzymology, Vol. 68, Academic Press, N.Y. (1979); T. Maniatis, E. F. Fritsch and J. Sambrook, Molecular Cloning: A Laboratory Manual, Cold Spring Harbor Laboratory Press, Cold Spring Harbor, N.Y. (1982); Catalog 1982-83, New England Biolabs, Inc.; Catalog 1982-83, Bethesda Research Laboratories, Inc. An example of how to construct the vectors employed in the subject methods is provided in the Experimental section, infra.

The subject methods find use in a variety of applications in which it is desired to introduce an exogenous nucleic acid into a target cell. As mentioned above, a feature of the subject methods is that a linear DNA vector employed in the subject methods is systemically administered to a multicellular organism that includes the target cell, i.e., the cell into which introduction of the nucleic acid is desired. By multicellular organism is meant an organism that is not a single-celled organism. The multicellular organism to which the vector is administered is an organism that includes a plurality of cells and is not a single-celled precursor thereof. Multicellular organisms of interest include plants and animals, where animals are of particular interest. Animals of interest include vertebrates, where the vertebrate is a mammal in many embodiments. Mammals of interest include; rodents, e.g., mice, rats; livestock, e.g., pigs, horses, cows, etc., pets, e.g., dogs, cats; and primates, e.g., humans. As the subject methods involve administration of the vector directly to the multicellular organism, they are in vivo methods of introducing the exogenous nucleic acid into the target cell.

The route of administration of the vector to the multicellular organism depends on several parameters, including: the nature of the vectors that carry the system components, the nature of the delivery vehicle, the nature of the multicellular organism, and the like, where a common feature of the mode of administration is that it provides for in vivo delivery of the vector components to the target cell(s) via a systemic route. Of particular interest as systemic routes are vascular routes, by which the vector is introduced into the vascular system of the host, e.g., an artery or vein, where intravenous routes of administration are of particular interest in many embodiments.

In certain embodiments, the linear DNA vector is administered in an aqueous delivery vehicle, e.g., a saline solution. As such, in many embodiments, the vector is administered intravascularly, e.g., intraarterially or intravenously, employing an aqueous based delivery vehicle, e.g., a saline solution.

The DNA vector is administered to the multicellular organism in an in vivo manner such that it is introduced into a target cell of the multicellular organism under conditions sufficient for expression of the nucleic acid present on the vector to occur. A feature of the subject methods is that they result in persistent expression of the nucleic acid present thereon, as opposed to transient expression. By persistent expression is meant that the expression of nucleic acid at a detectable level persists for an extended period of time, if not indefinitely, following administration of the subject vector. By extended period of time is meant at least I week, usually at least 2 months and more usually at least 6 months. By detectable level is meant that the expression of the nucleic acid is at a level such that one can detect the encoded protein in the mammal, e.g., in the serum of the mammal, at a level of at detectable levels at a therapeutic concentration. See e.g., the experimental section, supra. As compared to a control in which the dsDNA vector is administered as a closed, circular dsDNA molecule, e.g., a cc DNA molecule, protein expression persists for a period of time that is at least about 2 fold, usually at least about 5 fold and more usually at least about 10 fold longer following the subject methods as compared to a control.

A feature of many embodiments of the subject methods is that the above described persistent expression is achieved without integration of the vector DNA into the target cell genome of the host. As such, the vector DNA introduced into the target cells does not integrate into, i.e., insert into, the target cell genome, i.e., one or more chromosomes of the target cell. In other words, the vector DNA introduced by the subject methods does not fuse with or become covalently attached to chromosomes present in the target cell into which it is introduced by the subject methods.

In many embodiments, administration of the linear dsDNA vectors of the subject methods results in the production of concatamers of two or more vector molecules in the target cell. By concatamer is meant a molecule made up of the ligation of two or more dsDNA molecules, where the number of individual vectors that join or fuse together to produce a given concatamer in the subject methods may range from about 2 to 1000, usually from about 3 to 20 and more usually from about 3 to 10. As such, the resultant concatamers may range in size from about 2 kb to 100 kb, usually from about 4 kb to 20 kb. In certain embodiments, a feature of the subject methods is that the non-annealed plus and minus strands are administered to the host under conditions such that they anneal to each other in vivo following administration to the host. As such, in certain embodiments the plus and minus strands are administered as separate compositions to the host. Alternatively, they may be combined into a single composition immediately prior to administration in order to minimize annealing prior to administration. In yet other embodiments, they may be treated so as to limit or at least reduce the amount of a annealing that occurs prior to administration.

The particular dosage of dsDNA vector that is administered to the multicellular organism in the subject methods varies depending on the nature of vector, the nature of the expression module and gene, the nature of the delivery vehicle and the like. Dosages can readily be determined empirically by those of skill in the art. For example, in mice where the vectors are intravenously administered in a saline solution vehicle, the amount of vector that is administered in many embodiments typically ranges from about 1 to 200 and usually from about 10 to 50 μg.

The subject methods may be used to introduce nucleic acids of various sizes into the a target cell. Generally, the size of DNA that is inserted into a target cell using the subject methods ranges from about 0.5 to 100 kb, usually from about 2 to about 15 kb.

The subject methods may be employed to introduce a nucleic acid into a variety of different target cells. Target cells of interest include, but are not limited to: muscle, brain, endothelium, hepatic, and the like. Of particular interest in many embodiments is the use of the subject methods to introduce a nucleic acid into at least a hepatic cell of the host.

Utility

The subject methods find use in a variety of applications in which the introduction of a nucleic acid into a target cell is desired. Applications in which the subject vectors and methods find use include: research applications, polypeptide synthesis applications and therapeutic applications. Each of these representative categories of applications is described separately below in greater detail.

Research Applications

Examples of research applications in which the subject methods of nucleic acid introduction find use include applications designed to characterize a particular gene. In such applications, the subject vector is employed to introduce and express a gene of interest in a target cell and the resultant effect of the inserted gene on the cell's phenotype is observed. In this manner, information about the gene's activity and the nature of the product encoded thereby can be deduced. One can also employ the subject methods to produce models in which overexpression and/or misexpression of a gene of interest is produced in a cell and the effects of this mutant expression pattern are observed.

Polypeptide Synthesis Applications

In addition to the above research applications, the subject methods also find use in the synthesis of polypeptides, e.g. proteins of interest. In such applications, a linear DNA vector that includes a gene encoding the polypeptide of interest in combination with requisite and/or desired expression regulatory sequences, e.g. promoters, etc., (i.e. an expression module) is introduced into the target cell, via in vivo administration to the multicellular organism in which the target cell resides, that is to serve as an expression host for expression of the polypeptide. Following in vivo administration and subsequent stable integration into the target cell, the multicellualr organism, and targeted host cell present therein, is then maintained under conditions sufficient for expression of the integrated gene. The expressed protein is then harvested, and purified where desired, using any convenient protocol.

As such, the subject methods provide a means for at least enhancing the amount of a protein of interest in a multicellular organism. The term ‘at least enhance’ includes situations where the methods are employed to increase the amount of a protein in a multicellular organism where a certain initial amount of protein is present prior to in vivo administration of the vector. The term ‘at least enhance’ also includes those situations in which the multicellular organism includes substantially none of the protein prior to administration of the vector. As the subject methods find use in at least enhancing the amount of a protein present in a multicellular organism, they find use in a variety of different applications, including agricultural applications, pharmaceutical preparation applications, and the like, as well as therapeutic applications, described in greater detail infra.

Therapeutic Applications

The subject methods also find use in therapeutic applications, in which the vectors are employed to introduce a therapeutic nucleic acid, e.g., gene, into a target cell, i.e. gene therapy applications, to provide for persistent expression of the product encoded by the nucleic acid present on the vector. The subject vectors may be used to deliver a wide variety of therapeutic nucleic acids. Therapeutic nucleic acids of interest include genes that replace defective genes in the target host cell, such as those responsible for genetic defect based diseased conditions; genes which have therapeutic utility in the treatment of cancer; and the like. Specific therapeutic genes for use in the treatment of genetic defect based disease conditions include genes encoding the following products: factor VIII, factor IX, β-globin, low-density protein receptor, adenosine deaminase, purine nucleoside phosphorylase, sphingomyelinase, glucocerebrosidase, cystic fibrosis transmembrane regulator, α-antitrypsin, CD-18, ornithine transcarbamylase, arginosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, and the like, where of particular interest are human versions of these proteins. Cancer therapeutic genes that may be delivered via the subject methods include: genes that enhance the antitumor activity of lymphocytes, genes whose expression product enhances the immunogenicity of tumor cells, tumor suppressor genes, toxin genes, suicide genes, multiple-drug resistance genes, antisense sequences, and the like.

In certain embodiments, the subject methods also find use in gene targeting and repair applications analogous to the AAV vector based gene repair applications described in Hirata and Russell, “Design and packaging of adeno-associated virus gene targeting vectors,” J Virol. (May, 2000) 74(10):4612-20 and Inoue et al., “High-fidelity correction of mutations at multiple chromosomal positions by adeno-associated virus vectors,” J Virol. (September 1999) 73(9):7376-80, as well as WO 98/48005 and WO 00/24917, the disclosures of which are herein incorporated by reference. In these embodiments, the ssDNA vectors of the subject invention are employed in a manner analogous to the AAV vectors described in these publications.

The subject methods also find use in the expression of RNA products, e.g., antisense RNA, ribozymes etc., as described in Lieber et al., “Elimination of hepatitis C virus RNA in infected human hepatocytes by adenovirus-mediated expression of ribozymes,” J Virol. (December 1996) 70(12):8782-91; Lieber et al., “Related Articles Adenovirus-mediated expression of ribozymes in mice,” J Virol. (May 1996) 70(5):3153-8; Tang et al., “Intravenous angiotensinogen antisense in AAV-based vector decreases hypertension,” Am J Physiol. (December 1999) 277(6 Pt 2):H2392-9; Horster et al. “Recombinant AAV-2 harboring gfp-antisense/ribozyme fusion sequences monitor transduction, gene expression, and show anti-HIV-1 efficacy, Gene Ther. (July 1999) 6(7):1231-8; and Phillips et al., “Prolonged reduction of high blood pressure with an in vivo, nonpathogenic, adeno-associated viral vector delivery of AT1-R mRNA antisense,” Hypertension. (January 1997) 29(1 Pt 2):374-80. As such, the subject methods can be used to deliver therapeutic RNA molecules, e.g., antisense, ribozyme, etc., into target cells of the host.

An important feature of the subject methods, as described supra, is that the subject methods may be used for in vivo gene therapy applications. By in vivo gene therapy applications is meant that the target cell or cells in which expression of the therapeutic gene is desired are not removed from the host prior to contact with the vector system. In contrast, the subject vectors are administered directly to the multicellular organism and are taken up by the target cells, following which expression of the gene in the target cell occurs. Another important feature is that the resultant expression is persistent and occurs without integration of the vector DNA into the target cell genome.

Kits

Also provided by the subject invention are kits for use in practicing the subject methods of in vivo nucleic acid delivery to target cells, e.g., hepatic cells. The subject kits generally include a linear DNA vector molecule, generally in an aqueous medium. In addition, many of the subject kits include one or more modulatory oligonucleotides for use in the subject vectors. The subject kits may further include an aqueous delivery vehicle, e.g. a buffered saline solution, etc. In addition, the kits may include one or more restriction endonucleases for use in transfering a nucleic acid into the vector. In the subject kits, the above components may be combined into a single aqueous composition for delivery into the host or separate as different or disparate compositions, e.g., in separate containers. Optionally, the kit may further include a vascular delivery means for delivering the aqueous composition to the host, e.g. a syringe etc., where the delivery means may or may not be pre-loaded with the aqueous composition.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g. a piece or pieces of paper on which the information is printed, in the packaging of the kit, in a package insert, etc. Yet another means would be a computer readable medium, e.g. diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### Experimental

I. dsDNA Vectors

A. Materials and Methods

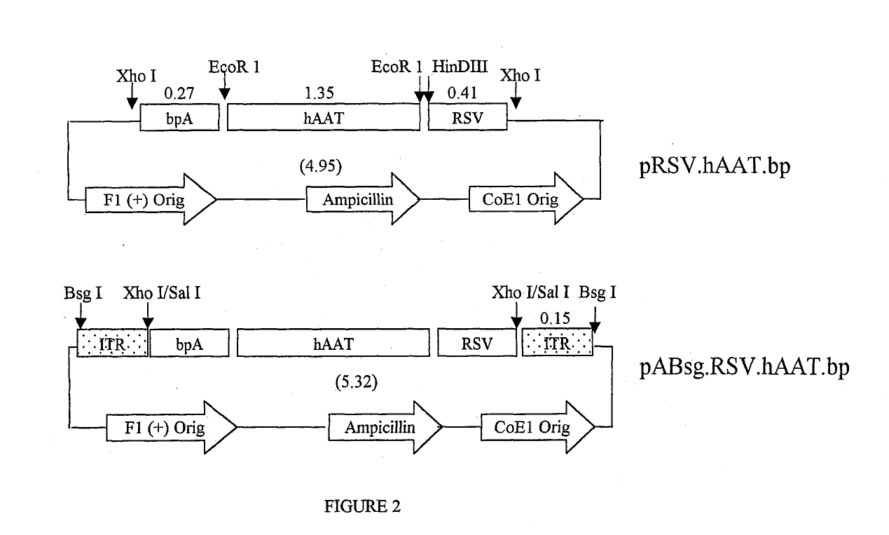

1. Construction of plasmids. Human alpha-1 antitrypsin (hAAT) cDNA with the poly A signal of bovine growth factor gene (bpA) was placed under the control of Rouse sarcoma virus long term repeat (RSV), and the expression cassette was inserted into the Xho I site in the multiple cloning site (MCS) of pBS.KS (Stratagene, La Jolla, Calif.). To prepare the recombinant AAV construct pABsg.RSV.haat.bpA, the Xho I released RSV.hAAT.bpA fragment was ligated to the pABsg.SENB vector prepared by Sal I digestion followed by dephosphorylation. The pABsg.SENB construct was derived from pALAPSN (Alexander I E et al., J V 68:8282, 1994) by building a Bsg I site into the outside boundary of each AAV ITR. The two constructs are shown in FIG. 2.

2. Preparation of plasmid and linear DNA. Plasmid DNA of the 2 constructs was used to transform the E. coli strain Sure Cells ( Stratagene, La Jolla, Calif.) and colonies from each construct were grown in LB broth. Large scale preparation of plasmid DNA was accomplished using a CsCl banding protocol. To prepare the linear DNA without AAV ITR termini (LDNA), plasmid DNA of pRSV.hAAT.bpA was digested with Xho I, followed by phenol-chloroform extraction and precipitated by ethanol alcohol. LDNA with AAV ITR termini (AAV LDNA) was prepared by digesting the pBsg.RSV.hAAT.bpA plasmid with Bsg I instead. The completeness of the AAV ITR was confirmed by DNA sequencing and by determination of the sizes of the Sma I-Bsg I and Sma I-Afl II sites, which located 4 bp away from each of the Sal I/Xho I sites. The completeness of the restriction digestion of the plasmid DNA was monitored in 0.8% of agarose gel. All of the resultant DNAs were dialyzed against TE for at least 24 hours with 2 changes of the buffer before use.

3. DNA delivery to mice. Eight to 10 week old female C57BL/6 mice were purchased from Taconi Farms Inc. (Germantown, N.Y.). All the animal procedures were performed following the National Institute of Health guidelines. Forty μg of each form of DNA, i.e. linear, circular, were dissolved in 2 ml of saline and delivered to the mouse via tail vein injection according to the method developed by Wolff, J. A. et al. (Zhang, G., et al., Human Gene Therapy, 10:1735-1737, 1999) and Liu, F., et al., (Gene Therapy 7: 1258, 1999).

4. ELISA quantitation of haat. After DNA delivery, mouse blood was collected periodically with a retro-orbital bleeding procedure. HAAT in mouse serum was quantitated by ELISA using a goat anti-hAAT antibody from DiaSorine (Stillwater, Minn.) (Kay, M. et al., 815, 1995).

5. Southern blot analysis of vector DNA structure. Mice were sacrificed 1 day, 5 and 10 weeks after DNA injection. Liver total DNA was prepared using a Salt Out procedure. The DNA was digested using different restriction enzymes which either did not cut (0-cutter, Bgl II), cut once (1-cutter, HinD III) or cut twice (2-cutter, EcoR 1) either vector (FIG. 2), separated by electrophoresis in 0.8% agarose and processed for Southern blot analysis according to Sambrook, J., et. al. (Sambrook, J., et. al., Molecular Cloning, 1989). A 1.35 kb EcoR 1-EcoR 1 fragment of hAAT cDNA from the pRSV.hAAT.bpA plasmid (FIG. 2) was used as the probe. DNA bands were quantitated using the Phospholmager of Bio-Rad Laboratories (Hercules, Calif.).

6. Statistics. Student t test was used to determine if the difference in transgene expression and in the amount of vector DNA remaining in different groups of mice were statistically significant.

B. Results

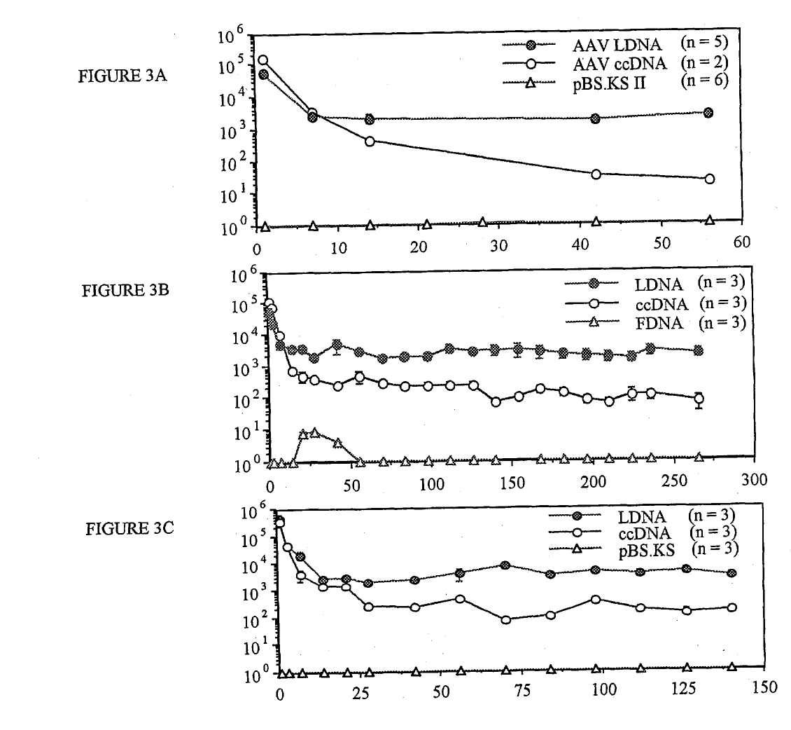

1. HAAT expression in mice. 40 μg of LDNA and ccDNA encoding a hAAT expression cassette (See FIG. 2) were delivered to each mouse via tail vein injection. This method of administration has been shown to target the mouse liver (Wolff J A et al., and Liu F et al.). After DNA administration, blood was collected periodically and serum hAAT was determined by ELISA (FIG. 3). One day after DNA delivery, the seral hAAT was 114,834±34,510 ng/ml and 50,705±18,128 ng/ml in mice receiving ccDNA (n=3) and LDNA (n=3), respectively (p>0.05). The transgene expression dropped more than 10 fold in one week in both groups, but mice receiving ccDNA continued to express 2 to 3 fold higher transgene than the mice received LDNA in this period of time. The ratio of transgene expression inversed beginning from week 2 when the seral hAAT in the LDNA group was 3,517.00±921.35 ng/ml while the ccDNA group expressed 4 fold lower of hAAT (698±89 ng/ml, p<0.05). The hAAT level continued to decline gradually in ccDNA group and was only 62.78±31.58 ng/ml at week 38. In contrast, seral hAAT in the LDNA group persisted above 1,500 ng/ml level, 5 to more than 30 fold higher than that in ccDNA group, for up to 38 weeks after DNA infusion (the experiment period). Mice receiving fragmented DNA expressed no detectable or background level of hAAT.

To confirm the observations of the first experiment, a repeated experiment was conducted using the same conditions with an increased number of mice. Six mice were used in each group until week 10 when 3 mice each were sacrificed for Southern blot analysis. Almost the same level of seral hAAT was detected in mice receiving ccDNA and LDNA (357,292±59,146 ng/ml vs. 405,715±174,415 ng/ml, (p>0.05) one day after DNA infusion and the hAAT level dropped greatly as seen in the first experiment. Again, the transgene expression stabilized above 2,000 ng/ml for up to 20 weeks (the experimental period) in the mice receiving LDNA, while seral hAAT was only one half of that of LDNA in week 2 (p<0.05) and the difference in seral hAAT between the two groups continued to increase with time and a 20 fold difference was seen at week 20 (2912±604 ng/ml vs. 148±37 ng/ml, n=3 each, p<0.05).

The following assay was performed to determine whether inclusion of AAV ITR in both linear DNA (AAV LDNA) and ccDNA (AAV ccDNA) would result in any enhancement in transgene expression. The 2 forms of DNA with AAV ITR flanking the hAAT expression cassette (FIG. 2) were injected into 2 groups of mice using the same conditions. A similar time course of transgene expression in the mice receiving both forms of DNA was demonstrated. The 5 mice receiving AAV LDNA expressed 56,585±10,109 ng/ml of hAAT one day after DNA injection while the 2 mice receiving AAV ccDNA expressed a average of 161,359 ng of hAAT/ml. The transgene dropped dramatically in both groups and the ratio of seral hAAT between the 2 groups inversed in week 2 when 2,095±590 ng of hAAT/ml in AAV LDNA group vs. 423 ng in AAV ccDNA group were detected. Seral hAAT in AAV LDNA was maintained above 1,600 ng/ml level in the 8 week period of the experiment while more than 100 fold lower of seral hAAT (20 ng/ml) was detected in AAV ccDND group at week 8. The above results are graphically represented in FIG. 3.

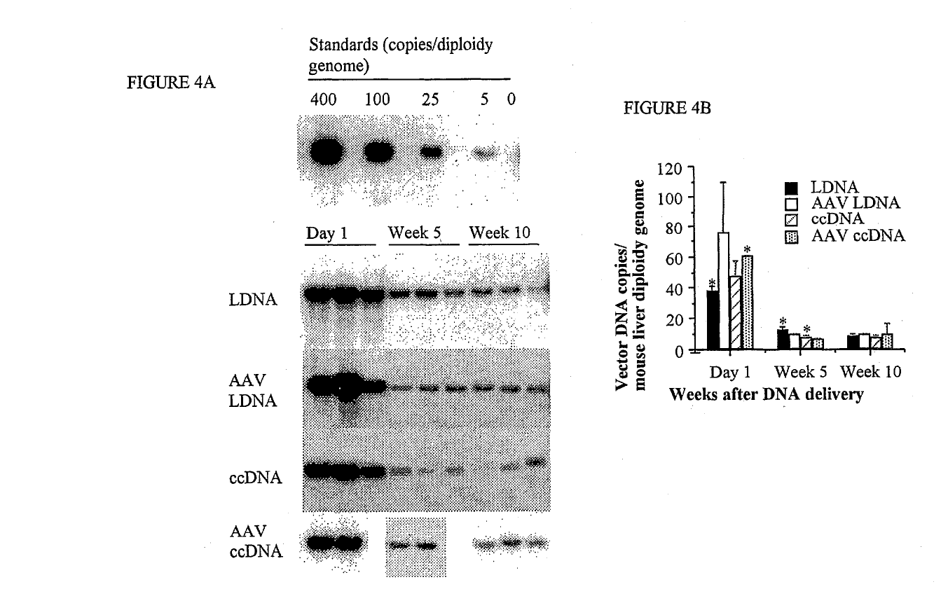

2. Persistence of vector DNA in mouse livers. A series of standards including 5, 25, 100, and 400 copies of hAAT cDNA per mouse diploidy genome resulted in a linear dose-signal intensity relationship using PhosphoImage quantitation (FIG. 4; Y=−321.38+51.624X, r\hat{ }2=0.993). The vector DNA remained in mouse livers one day after DNA delivery was 38±4, 76±33, 48±9, and 61 copies per mouse liver diploidy genomic DNA from livers of mice receiving LDNA (n=3), AAV LDNA (n=3), ccDNA (n=3), and AAV ccDNA (n−2), respectively. The vector DNA amount was statistically significantly higher in the AAV ccDNA group than that in the LDNA group (p<0.05) one day after DNA injection. The vector DNA amount detected 5 weeks later was 68 to 88 percent of that in day 1 and significantly more vector DNA was seen in LDNA group (12±1 copies/liver diploidy genome, n=3) than in ccDNA group (8±1 copies/liver diploidy genome, n=3, p<0.05). The amount of vector DNA remained almost unchanged after 5 more weeks and the difference in the copy number of vector DNA per diploidy liver genomic DNA among all groups was statistically insignificant. The difference in vector copy number per liver diploidy genome between LDNA and ccDNA, and AAV LDNA and AAV LDNA in weeks 5 and 10 was no more than 50 percent, while the difference in hAAT expression between them was more than 5 fold in both time points, suggesting that the difference in vector DNA amount is not responsible for the difference in transgene expression between the groups and a factor other than the vector DNA amount is responsible for the difference in transgene expression.

3. Formation of large concatamers by LDNA and AAV LDNA in mouse liver. Southern blot analysis was conducted to understand the molecular mechanism underlying the difference in the transgene expression mediated by different forms of DNA vectors. When the liver total DNA from mice receiving LDNA digested with Bgl II, which does not cut the vector DNA, and probed with the hAAT cDNA, a strong band at around 23 kb was seen in all 9 DNA samples of mice sacrificed 1 day, 5 and 10 weeks after DNA infusion. As judged from ethidium-bromide-stained gel, this band is composed of DNA species which migrated the slowest, suggesting that they represented a population of large concatamers formed by the linear vector DNA fragments. The linear DNA fragment used for injection in this experiment and detected by the hAAT cDNA probe is 2.03 kb in size, and Bgl II digestion of the mouse genomic DNA should result in 3.0 kb fragments in average. If there is no concatamerization, and even if individual vector DNA fragment was integrated, a DNA smear composed of DNA species 5 kb in average size should be seen. The formation of the large concatamers by the linear vector DNA fragments was further confirmed by Southern blot using the same group of DNA samples digested with the 1-cutter HinD III, which cuts through the hAAT expression cassette once. The band around 23 kb disappeared but a DNA ladder composed of DNA fragments raging from around 2 kb to larger than 12.3 kb shows up. All the bands align very well with the bands in the HinD III-cut reference DNA lane, which was the product of religation of the same 2 DNA fragments result in from Xho I digestion of pRSV.hAAT.bpA and delivered to this group of mice. Clearly, these vector DNA bands are composed of a different number of DNA fragments which have been fused together in the mouse liver. The signal of the large concatamers can be seen in all 3 day 1 samples and the signal intensity of this species of vector DNA decreases only slightly from week 5 to week 10, indicating that mouse liver can form the concatamers from small DNA fragments very efficiently and that the large concatamers are relatively stable. There are several DNA bands smaller than 23 kb in the 3 day 1 DNA samples but most of them are not seen in week 5 and 10. A clear band slightly larger than 1.35 kb, and 2 very faint bands around 3 kb, can be seen in the week 5 and 10 samples. They probably represent products of religation-circularization of the DNA fragments because they disappeared in the HinD III cut blot. Strong degraded vector DNA signals can be seen in the 3 day 1 samples but are absent in the mice in the 2 later time points. Similar band patterns are demonstrated in the Southern blots using the DNA samples from mice receiving linear DNA with AAV ITR (AAV LDNA) and digested with either the 0-cutter Bgl II or the 1-cutter HinD III, indicating that AAV LDNA also formed large concatamers in mouse livers. The DNA ladder produced by HinD III digestion in this group of samples differs only slightly from that of LDNA group—each band migrates slightly slower than the corresponding bands in the reference DNA lane—apparently because each band contains one or more of the 145 bp AAV ITR. Three DNA bands from 2 to slightly larger than 3 kb are seen in the Southern blot using 1 randomly selected week 5 sample from each of LDNA and AAV LDNA groups and digested with both HinD III and Sca I, which made a single cut in the bacterial DNA backbone. This pattern agrees with the hypothesis that the orientation of either DNA fragment is random in the large concatamers.

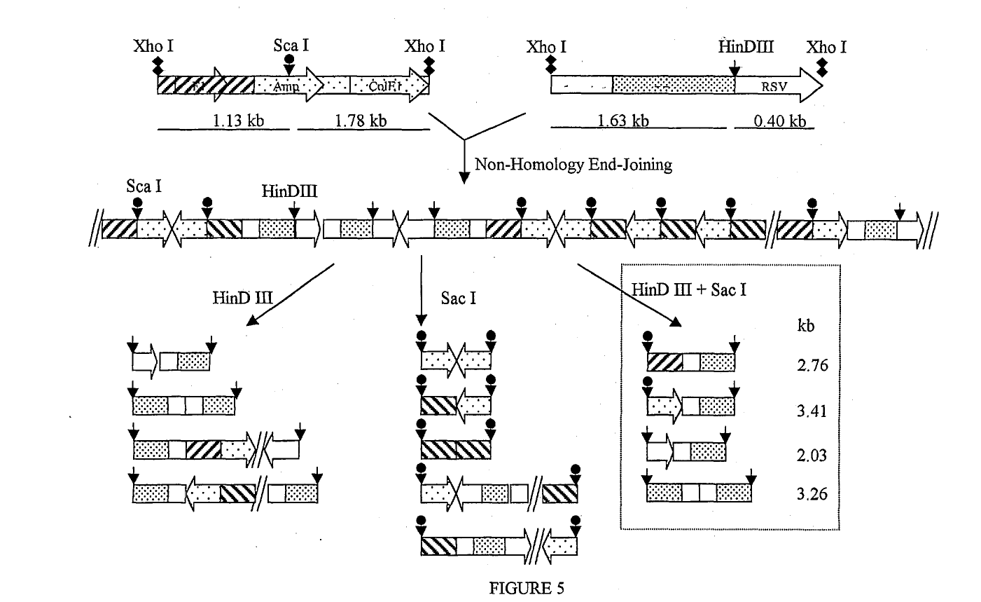

The hypothesis of formation of the large concatamers by 2 DNA fragments, produced by Xho I-digestion of pRSV.hAAT.bpA in mouse liver is schematically illustrated in FIG. 5. As demonstrated in the Southern blot, the 2 DNA fragments are fused together randomly. Different numbers of either fragments of hAAT expression cassette or the bacteria backbone are fused together in either orientation and then fused to each other. Digestion of concatamers with the 1-cutter HinD III and probed with hAAT cDNA resulted in a DNA ladder composed of bands of different sizes, each containing different numbers of bacteria backbone fragments flanked with either the RSV promoter or the hAAT cDNA plus bpA sequence of the hAAT expression cassette divided by HinD III digestion. A DNA ladder is also produced if Sca I, which cuts the bacteria backbone once, is used. In this condition, each band will be composed of different number of hAAT expression cassette flanked with either part of the bacteria backbone divided by Sac I. Digestion with both HinD III and Sca I result in 4 DNA bands. Only the DNA bands containing the hAAT cDNA detectable by the hAAT cDNA probe are shown in this scheme. Using pBS.KS to probe the same blot should produce similar DNA ladders with only slightly differences. This scheme should also illustrate the formation of concatamers by the 2 DAN fragments produced by Bsg I digestion of pABsg.hAAT.bpA. The only difference is that the hAAT expression cassette is flanked with AAV ITR termini and the size of each band in the DNA ladder will increase slightly.

4. Plasmid vectors remained intact in mouse livers. In the blot using the DNA samples from ccDNA group digested with the 0-cutter BgI II, 2 clear bands, one larger and one smaller than the full length vector (4.95 kb), can be seen in all 9 samples of 3 time points. A third band larger than 12.3 kb is obvious in the 3 day 1 samples but only barely detectable in the 6 week 5 and 10 mice. These bands appear to represent super coiled and aggregates of 2 or more copies of the plasmid in that only one band of the full length plasmid showed up in blot with the DNA digested with the 1-cutter HinD III and all the three bands seen in the BgI II digested DNA blot disappeared. This hypothesis is supported by the observation that vector DNA in one week 5 sample demonstrate the same migration pattern as the ccDNA spiked with untreated mouse genomic DNA and digested with BgI II. Obviously, 5 or 10 weeks after delivered to mouse livers, the ccDNA are intact and showed no detectable integration or concatamerization. Several extra bands and signal of degraded vector DNA were seen in the 3 day 1 samples but disappeared in all weeks 5 and 10 livers.

Only one single band is seen in all 7 liver DNA samples from mice receiving AAV ccDNA digested with either BgI II or HinD 111. Again, there is no detectable integration and concatamerization. Slight signal of degraded vector DNA is demonstrated in the 2 day 1 samples but can not be seen in other 5 mice sacrificed at later time points.

C. Discussion

The above results demonstrate that linear DNA can express sustainable and high levels of a transgene in mouse liver. More than 1.5 μg/ml of transgene hAAT was expressed for at least 9 months post linear DNA injection. In contrast, a comparable level of hAAT was expressed only transiently in the mice receiving a same amount of circular plasmid DNA and the transgene expression attenuates gradually, a pattern typical to the closed circular plasmid DNA as demonstrated in liver (Zhang G et al., 1997; Zhang C et al., 1999; Liu G, 1999; Miao C et al., J Mol Med 2000) as well as other organs (Jenkins R G et al., (+Hart S L) Gene Ther 7:393, 2000; Zou S.-M et al., (Behr) J Gene Med 2:128, 2000) in this (Miao C et al., J Mol Med 2000; Yang S et al., Nat Med 2000) and other laboratories (Zhang G et al., 1997; Zhang C et al., 1999; Liu G, 1999; Jenkins R G et al., (+Hart S L) Gene Ther 7:393, 2000; Zou S.-M et al., (Behr) J Gene Med 2:128, 2000).

The above results also demonstrate that formation of large concatamers is responsible for the long lasting and high level of transgene expression in mice receiving linear DNA. The large concatemers were seen only in the liver of mice receiving linear DNA while closed circular plasmid remained intact without detectable concatamerization and integration in mouse liver. The large concatamers were formed in day 1 of DNA injection and they remained relatively stable from day 1 through week 5 and week 10 when most of the smaller size of vector DNA seen in day 1 disappeared and the large concatamers became almost the exclusive vector DNA species. The difference in vector DNA amount between the livers of the 2 groups of mice is small and can not explain the more than 5 fold difference in transgene expression between them from week 5 to week 10 post DNA injection. It appears that a transgene in the large concatamer is more favorable for gene expression than in the closed circular form.

Interestingly, inclusion of wild type AAV ITR flanking the transgene expression cassette in both linear DNA and closed circular plasmid did not result in a difference in either pattern of transgene expression or the DNA structure alteration. Linear DNA with wild type AAV ITR termini demonstrated a similar transgene expression time course and formed large concatamers. Likewise, intact plasmid containing AAV ITR also expressed the transgene transiently, remained intact, and showed no detectable concatamerization or integration. The palindromic sequence of the AAV ITR is an effective component responsible for integration into host genome and the intermolecular recombination integration of the viral genome (Yang J et al., JV 73:9468, 1999). However, our data indicate that AAV ITR is not necessary in the high efficiency of large concatamer formation. This finding indicates that the requirements for efficient large concatamer formation are simple. No virus or helper protein from outside the cells is involved and no special DNA sequence within the vector is required. The linear DNA is the only component needed. Furthermore, DNA elements capable of optimizing the transgene expression, such as those enhancing the transcription, stabilizing the transgene, and on-off switching of the gene expression, can be conveniently included in a same concatamer, as long as they are not restrictive in their location and orientation relative to the transgene expression cassette. In addition to the other advantages of nonviral vectors over the viral vectors as discussed earlier, linear DNA technology is able to use larger DNA fragments without the size limit posed to AAV virus, and have more freedom to include DNA elements with versatile functions. Artificial chromosomes can be built in vivo using this linear DNA technology by using appropriate centromere, telomere and replication origins.

Linear DNA technology described in this study provides a novel solution to overcome one major obstacle, the short duration of transgene expression, for the clinical use of nonviral vectors. As compared to the currently employed vectors, e.g., the retroviruses; the AAV viruses; and the transposon; that mediate insertion of transgene to the host genome and result in life-long gene expression, linear DNA technology involves no virus and no foreign protein so that there will be no toxicity or immune response.

II. ssDNA Vectors

A. Methods

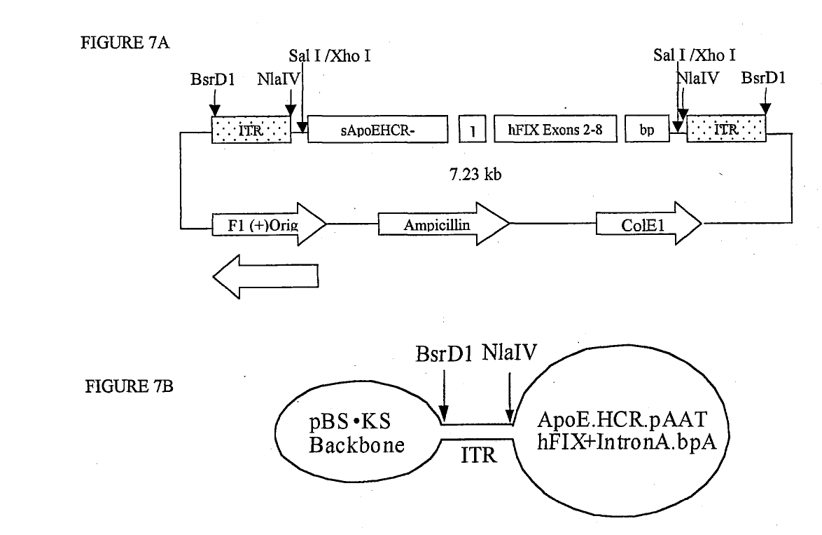

1. Preparation of constructs and ssDNA. The construct pA.sApoEHCR.phAAT.hFIX+intronA.bpA was derived from construct pBS.sApoEHCR.phAAT.hFIX+intronA.bpA which is described elsewhere (Miao C. H. et al., Molecular Therapy (in press)). Briefly, the enhancer-promoter region was composed of a 711 bp fragment of human ApoE locus control region (HCR) and a 408 bp fragment of human alpha-1 antitrypsin promoter (phAAT). The human factor IX (hFIX) cDNA contained a part of the 1st intron (1.4 kb) between the exons 1 and 2, and the poly A signal (0.3 kb) derived from bovine growth factor (bpA) (See FIG. 7). To add AAV ITRs to flank the hFIX expression cassette, the poEHCR. phAAT.hFIX+intronA.bpA fragment was isolated using 2 Spe I sites outside the expression cassette, which was then inserted into the Spe I cut pBS.KS II (+) (Stratagene, La Jolla, Calif.). The expression cassette was removed using 2 Xho I sites, and ligated to a modified Sal I digested vector pALAPSIN (Alexander I. E. et al., J. Virol., 68:8282-8287. 1994). A BrsD1 site was built into the outside boundary of each ITR in the pALAPSIN vector. The ITR flanked ApoEHCR.phAAT.hFIX +intronA.bpA expressing cassette was then removed using 2 EcoR1 sites outside the 2 BrsD1 sites and ligated to EcorR1 cut pBS.KSII(+). To produce phagemid ssDNA encoding the ApoEHCR.phAAT.hFIX+intronA.bpA expression cassette, the plasmid was used to transform Sure Cells and grown in 2XYT in the presence of VCSM13 (Stratagene, La Jolla, Calif.) according to the manufacturer's instruction and the phagemid ssDNA was recovered from the medium by PEG precipitation (Molecular Cloning, Sambrook, et al., 1989). The phagemid ssDNA was dissolved in 1 x restriction buffer, heated at 95° C. for 5 minutes and slowly cooled to room temperature, allowing the 2 complementary AAV ITR, as well as the complementary BsrD1 and NIa IV, recognition sequences to anneal. The annealed phagemid ssDNA was then digested with either BsrD1 or NIaIV to produce ssDNA with or without AAV ITR (FIG. 7). The digested phagemid DNA was separated by electrophoresis in 0.8% agarose gel and the gel band containing the desired ssDNA was dissected. The ssDNA was electro-eluted, precipitated with ethanol and dialyzed against TE before use. The complementary single-strand DNA was produced by either switching the orientation of the expression cassette or by placing the expression cassette into the pBS.KS (−) vector (Stratagene, La Jolla, Calif.).

2. DNA delivery to mice via tail vein injection. All animal procedures were conducted according the Guidelines of National Institute of Health. Forty μg of plus strand and minus strand of ssDNA, or a mix of 20 μg of each of the 2 strands, were dissolved in 2.0 ml of saline and delivered to 8 to 10 week old C57BL/6 mice (Taconic Farms Inc., Germantown, N.Y.) via the tail vein injection technique developed by Wolff, J. A., et al. (Zhang, G., et al., Human Gene Therapy, 10:1735-1737, 1999), and Liu, F. et al. (Gene Therapy, 6:1258, 1999). To destroy any secondary structure, all ssDNA was heated to 95° C. for 5 minutes and chilled immediately on ice before injection. For the mix of the 2 strands of ssDNA, minus and plus strand samples were heated and chilled separately and mixed immediately before injection to minimize the annealing of the 2 strands prior to injection.

3. ELISA determination of hFIX. Mouse blood was periodically collected after DNA delivery with a retro-orbital bleeding technique. Seral hFIX was determined by ELISA using a rabbit anti-human FIX antibody (Dako Co., Carpinteria, Calif.) (Walter, J. et al., PNAS USA, 93:3056-3061, 1996).

B. Results

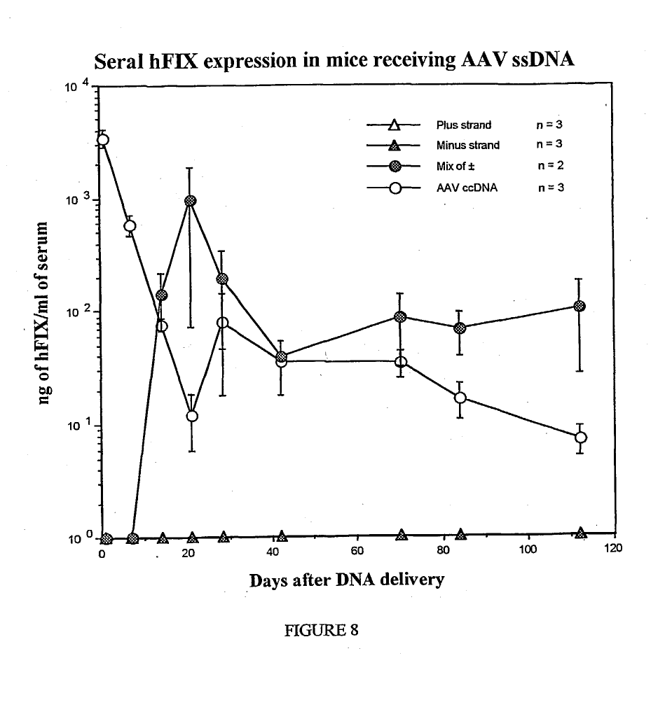

1. ELISA determination of hFIX in the mice receiving AAV ssDNA. In the first experiment, 3 mice receiving 40 μg of double-stranded circular plasmid DNA expressed an average of 3,425±1075 ng of hFIX/ml 1 day after DNA injection (FIG. 8). The serum hFIX levels dropped to 76±10, and 7±5 ng/ml at week 2 and 10, respectively. In contrast, two mice receiving a mix of 20 μg of plus and minus strands of AAV ssDNA did not express detectable hFIX until week 2 when 68.31 and 215.31 ng/ml was detected. Importantly, hFIX gene expression remained stable for at least 16 weeks (time of experiment). All the mice receiving plus (n=3), or minus (n=3) AAV ssDNA alone showed no detectable hFIX throughout the experiment.

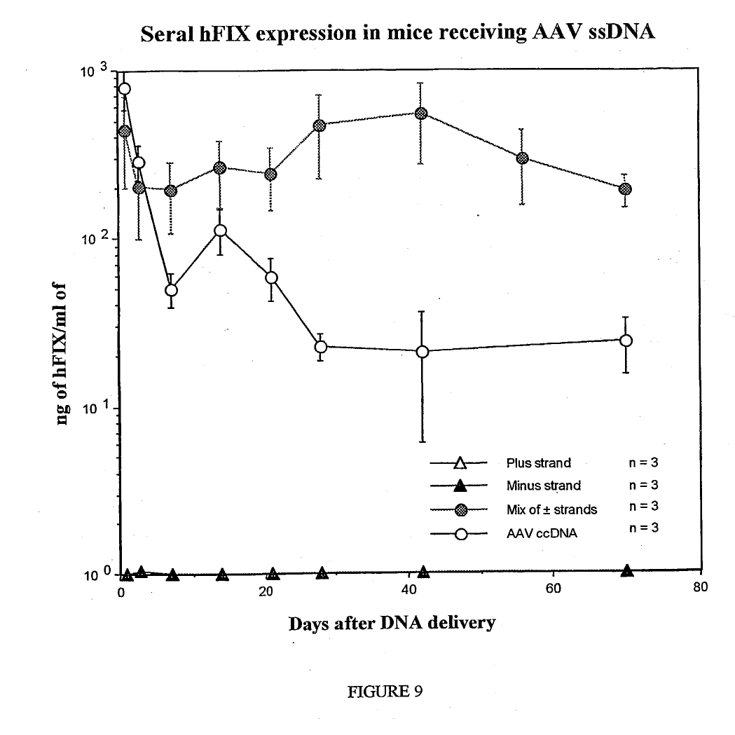

In a repeat experiment using the same conditions, a similar hFIX expression pattern was demonstrated in both groups of mice. In the plasmid DNA group (n=3), the hFIX expression peaked at 795±382 ng/ml 1 day after DNA injection and dropped to below 100 ng/ml level in 2 weeks, and continued to decline over time. In contrast, the 2 mice receiving a mix of the two strands of AAV ssDNA expressed about 200 ng of hFIX/ml for up to 10 weeks, the latest time point when this report was prepared. Again, all the mice receiving either plus (n=3), or minus (n=3) strand of AAV ssDNA along showed no detectable hFIX through the experiment. See FIG. 9.

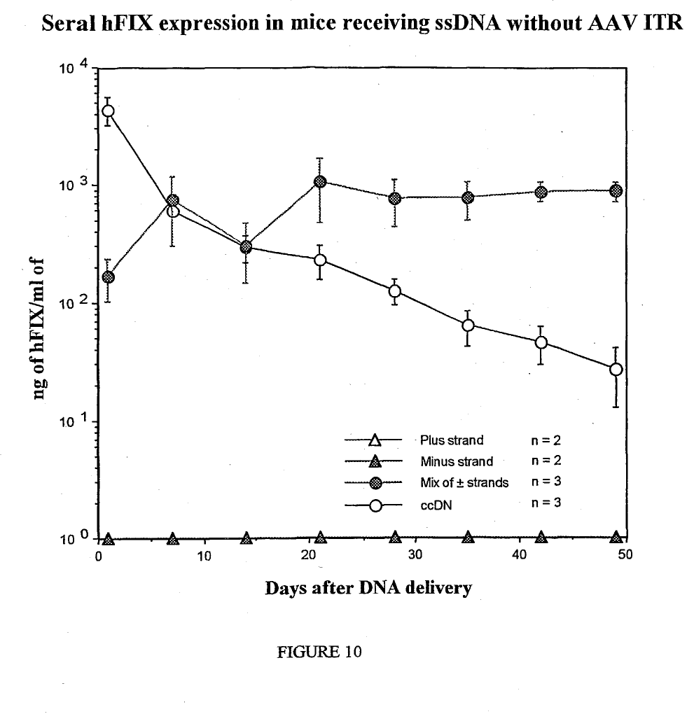

2. ELISA determination of hFIX in the mice receiving ssDNA without the AAV ITRs. To determine if the AAV ITRs were required for mediating the sustained expression for single-stranded DNA, a mix of plus and minus strand of the same expression cassettes without AAV ITRs were infused into mice. This experiment demonstrated a similar hFIX expression pattern in mice. Three mice receiving double stranded plasmid DNAs expressed an average of 4,408±1170 ng/ml 1 day after DNA injection, but dropped to 296±76 ng/ml at week 2, and decreased further to 27±14 ng/ml at week 9. However, the 3 mice receiving a mix of plus and minus strands of ssDNA expressed an average of 127±68 ng/ml 1 day after DNA treatment, but the hFIX went up to 452±443 ng/ml within a week and serum hFIX was maintained at this level until week 9 (length of the experiment). At week 9, the serum hFIX levels in the ssDNA mix group was 17 fold higher than that in the plasmid DNA group (p<0.01). Each strand administered individually did not result in gene expression. See FIG. 10.

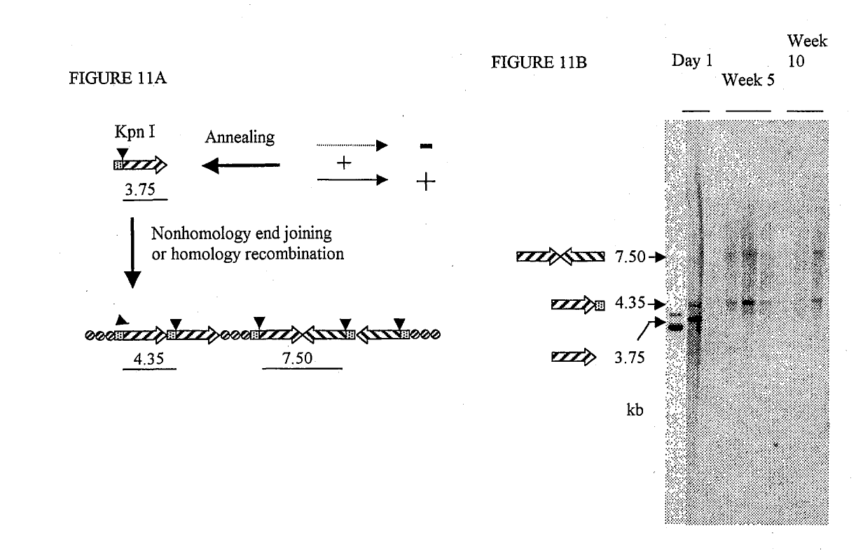

3. Liver total DNA was prepared from mice 1 day, 5 weeks and 10 weeks after receiving 20 micrograms of each of the plus and minus strands of ssDNA. Twenty micrograms of the total DNA was digested with restriction enzyme Kpn I, which cut the expression cassette once, and processed for Southern blot analysis. The probe used was composed of 2 PCR DNA fragments produced using the A.sApoEHCR.hAATp.hKIX+intran A.bpA as template and was specific for hFIX without cross-hybridizing to mouse FIX. The results are provided in FIG. 11.

C. Discussion

The results here demonstrate that hepatic delivery of single-stranded plus and minus genomes can result in persistent and therapeutic levels of transgene products such as human factor IX (at least 16 weeks). Though not wishing to be bound by a particular theory, it appeared that a transcriptionally active form of DNA was formed by in vivo annealing of plus and minus strands of ssDNA. Two findings support this hypothesis. First, transgene expression was detected only in the mice receiving a mix of plus and minus strands of ssDNA but not in mice receiving plus or minus ssDNA alone. Second, in contrast to the infusion of double-stranded plasmid DNAs in which gene expression was maximal within the first 24 hours, single-stranded DNA mediated gene expression slowly increased over a period of at least a week and was then sustained.

The finding that ssDNA is an efficient form of DNA to express a sustainable high level transgene in mouse livers is important for the development of nonviral DNA delivery systems for gene therapy. Because the mass of a ssDNA is only about one quarter to one third of a ccDNA 5 to 7 kb vector, the single-stranded molecules form much smaller vector particles than the ccDNA. In liver, the size of DNA particle is a critical factor in determining the transduction efficiency of the vector, in that any vector in the circulation has to pass the sinusoid pores, with a diameter of 80 nm also, to transfect hepatocytes. The smaller the size of the vector, the more efficient to pass the sinusoid pores, and consequently the higher transduction efficiency.

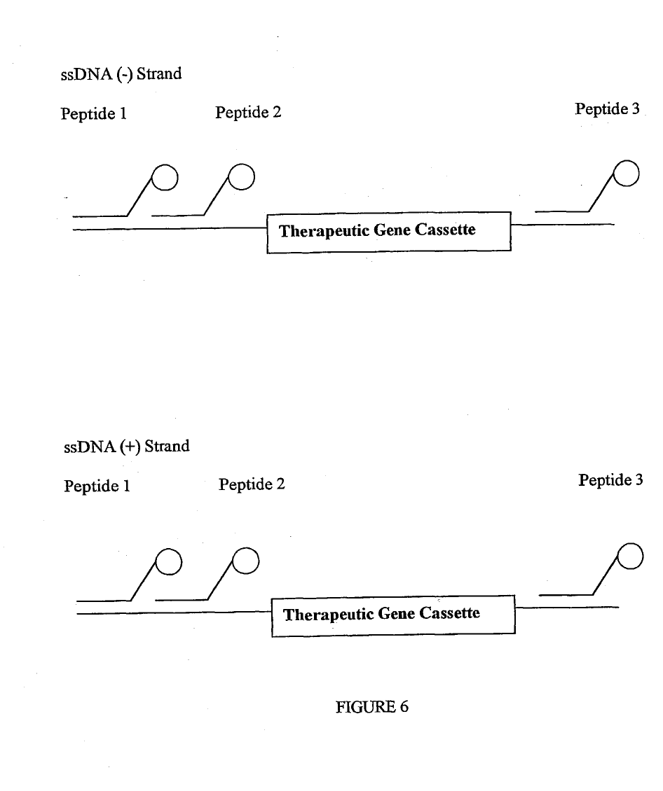

The use of single-stranded DNAs is also important because of the possibility of using small complementary oligos attached to functional peptides. See e.g., FIG. 6. These may include cell specific ligands, nuclear localization signals, and/or endosomal lysis, all of which may enhance gene transfer.

Mice receiving the ssDNA with the same hFIX expression cassette but lacking the AAV ITR expressed a high and sustainable level of hFIX, suggesting that AAV ITR did not play an important role expressing the transgene.

The above data clearly demonstrates that ssDNA, with or without an AAV ITR, is better than the control ccDNA to mediate sustained transgene expression in vivo.

It is evident from the above results and discussion that an improved method of transferring a nucleic acid into a target cell is provided by the subject invention. Specifically, the subject invention provides for a highly efficient in vivo method for nucleic acid transfer which does not employ viral vectors and does not require target cell genome integration and yet provides for persistent high level gene expression and therefore provides many advantages over prior art methods of nucleic acid transfer. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for introducing a nucleic acid into a target cell of a vascularized multi-cellular organism, said method comprising:
systemically administering to said vascularized multi-cellular organism a non-viral linear DNA vector comprising said nucleic acid to introduce said nucleic acid into said target cell.

2. The method according to claim 1, wherein said linear DNA vector is a dsDNA molecule.

3. The method according to claim 1, wherein said linear DNA vector is made up of non-annealed complementary plus and minus strands.

4. The method according to claims 1, 2 or 3, wherein said administering is intravenous.

5. The method according to claims 1, 2, 3 or 4, wherein said vascularized multi-cellular organism is a mammal.

6. The method according to any of the preceding claims, wherein said nucleic acid encodes a protein.

7. The method according to any of the preceding claims, wherein said linear DNA is hybridized to at least one modulatory oligonucleotide.

8. The method according to claim 7, wherein said modulatory oligonucleotide comprises a ligand domain stably associated with an oligonucleotide domain.

9. The method according to any of the preceding claims, wherein said target cell is hepatic cell.

10. The method according to any of the preceding claims, wherein said nucleic acid is an expression cassette encoding a protein.

11. The method according to claim 10, wherein said method results in persistent expression of said protein.

12. The method according to any of the preceding claims, wherein a concatamer is produced said target cell.

13. A DNA vector comprising:
(a) a linear DNA; and
(b) a modulatory oligonucleotide comprising a ligand domain stably associated with an oligonucleotide domain, wherein at least a portion of said oligonucleotide domain is hybridized to said linear dsDNA.

14. The vector according to claim 13, wherein said linear DNA is a dsDNA molecule.

15. The vector according to claim 13, wherein said linear DNA is made up of non-annealed complementary plus and minus strands that are capable of annealing into a dsDNA molecule.

16. The vector according to any of claims 13, 14 or 15, wherein said vector further comprises at least one restriction site.

17. The vector according to claim 16, wherein said at least one restriction site is part of a multiple cloning site.

18. The vector according to any of claims 13 to 17, wherein said vector further comprises a nucleic acid encoding a protein.

19. The vector according to claim 18, wherein said nucleic acid is part of an expression cassette.

20. The vector according to any of claims 13 to 19, wherein said ligand domain is covalently attached to said oligonucleotide domain.

21. A kit for producing a DNA vector, said kit comprising:
(a) a linear DNA molecule; and
(b) a modulatory oligonucleotide comprising a ligand domain stably associated with an oligonucleotide domain, wherein at least a portion of said oligonucleotide domain is capable of hybridizing to said linear dsDNA molecule under stringent conditions.

22. The kit according to claim 21, wherein said linear DNA molecule is a dsDNA molecule.

23. The kit according to claim 21, wherein said linear DNA molecule is made up of non-annealed complementary plus and minus strands that are capable of annealing into a dsDNA molecule.

24. The kit according to any of claims 21 to 23, wherein said kit further comprises a physiologically compatible aqueous delivery vehicle.

25. A pharmaceutical composition comprising
(a) a vector according to any of claims 13 to 20; and
(b) a pharmaceutically acceptable carrier, diluent and/or adjuvant.

26. The composition of claim 25 for the delivery of a nucleic acid into a target cell or into a target organism.

27. The composition of claims 25 or 26 for therapeutic applications.

28. The composition of claim 27 for gene therapy or nucleic acid vaccination.

29. The composition of any of claims 24 to 28 for systemic administration.
